(19) United States
(12) Patent Application Publication
(10) Pub. No.: US 2009/0216208 A1
(43) Pub. Date: Aug. 27, 2009

(54) Ostomy Carrier Device

(76) Inventor: Henrik Leisner, Gentofte (DK)

Correspondence Address: JACOBSON HOLMAN PLLC, 400 SEVENTH STREET N.W., SUITE 600, WASHINGTON, DC, 20004, US

(21) Appl. No.: 12/083,226

(22) PCT Filed: Oct. 10, 2006
(86) PCT No.: PCT/DK2006/000571

§ 371 Date: Mar. 11, 2009

(30) Foreign Application Priority Data
Oct. 10, 2005 (DK) PA 2005 01418

(51) Int. Cl. A61F 5/449; B23K 26/20

(52) U.S. Cl. 604/344; 219/121.64

(57) ABSTRACT

The invention relates to an ostomy carrier device comprising, a base plate for securing the carrier device to a user's skin around a stoma, the base plate having an opening for receiving the stoma; a circumferential flange adapted for connection with and removal of an ostomy collecting bag, the circumferential flange having an opening for receiving a stoma; at least one connecting ring arranged between the circumferential flange and the base plate; and at least one point on said connecting ring being radially displaceable from a neutral position, where no force is applied to the connecting ring, to a displaced position when force is applied to said at least one point, and that when said at least one point is in the displaced position said at least one point will move towards the neutral position when no force is applied to said connecting ring. This reduces the risk of unwanted leakage and/or inconvenience to the user.

The present invention relates to an ostomy carrier device comprising a flexible adhesive base plate, as well as a circumferential mounting flange made of a flexible material and being adapted for removable and adhesive connection with a coupling element of an ostomy collecting bag, the carrier device and the collecting bag making up a collecting system.

In collecting systems, the base plate of the carrier device must be able to remain attached to the user over a long period of time, for example up to 8-10 days. During this whole period of time, the carrier device must be capable of withstanding deformation due to the user's movements, washing, exposure to bag replacements, etc.

One such carrier device is known from the applicants own application EP 1 280 483 which discloses an ostomy carrier device with a base plate and a flexible circumferential flange the inner portion of which is more rigid than the outer portion. In order to ensure a flat device the circumferential flange has a low axial dimension. The effect is that the circumferential flange is locked for relative radial movement relative to the base plate.

EP 0 686 381 A1 discloses an ostomy applicance having an adhesive face and a first and second annular film. The outer portions of the annular films are sealed to a relatively stiff flange ring and between the inner portions is there provided a soft and easy deformable adhesive material, which is extrudable by finger pressure and which is fluid-absorbing, skin-friendly and capable of adhering to dry and moist skin surfaces.

Other ostomy carrier devices may be seen in EP 276 042, WO 99/26565 and WO 96/38106.

It is an object of the present invention to provide an ostomy carrier device wherein the base plate and the circumferential flange are free to move relative to each other, especially in the radial direction.

It is a further object of the present invention to provide an ostomy carrier device wherein the inner surfaces of the carrier device are as smooth as possible such that solid matter is not trapped inside the device, which is to be used for several days.

Furthermore, it is an object to provide a carrier device, which ensures a safe and reliable bond between the carrier device and the attached collecting bag and yet provides a high degree of wearing comfort.

Additionally, it is an object of the present invention to provide an ostomy carrier device wherein its elements may be welded together, especially by means of laser welding.

According to a first aspect the present invention relates to an ostomy carrier device comprising, a base plate for securing the carrier device to a user's skin around a stoma, the base plate having an opening for receiving the stoma; a circumferential flange adapted for connection with and removal of an ostomy collecting bag, the circumferential flange having an opening for receiving a stoma; at least one connecting ring arranged between the circumferential flange and the base plate; and at least one point on said connecting ring being radially displaceable from a neutral position, where no force is applied to the connecting ring, to a displaced position when force is applied to said at least one point, and that when said at least one point is in the displaced position said at least one point will move towards the neutral position when no force is applied to said connecting ring.

This advantageously provides for an ostomy carrier device wherein the base plate and the circumferential flange is allowed to be radially displaced to one another reducing the affect of radially forces on the ostomy carrier or the thereon applied ostomy bag and thereby reducing the risk of unwanted leakage and/or inconvenience to the user.

In one embodiment of such an ostomy carrier device according to the invention, the at least one connecting ring is formed of an elastic material, having a resilience allowing the at least one point to move from the displaced position towards the neutral position when a force is no applied to the connecting ring.

Such an elastic material may for example be an elastomer as will be discussed, or any other known elastic material which have the characteristic that it may be deformed by application of a force, for example squeezing building up an energy potential under the force application, and will return to its original shape when no force is applied to it, thereby releasing said build up energy potential. Such other known materials which are elastic or acts elastic may for example be metals, like steel and aluminium or a foam as will be discussed later.

In another embodiment of an ostomy carrier device according to the invention the at least one connecting ring is radially flexible. Thus flexible connecting rings, which allows the at least one point to move from the displaced position towards the neutral position when no force is applied to the connecting ring may be provided. Such connecting rings formed of a flexible material, e.g. a thin metal ring showing spring like characteristics, allows the tension and build up of potential energy when moving a point on the ring to a displaced position. When the tension is released the potential energy is also released and the point on the ring will move back towards its original, neutral position.

Such flexible connecting rings may not only be made of metal, but can be formed of many other materials, e.g. polymers. The material should preferably be formed relative thin to allow for easy flexibility, allowing deformation towards the displaced positioned where the resilience of the material would bias the material to the original, neutral position.

In a further embodiment the at least one connecting ring has an axial dimension extending between the circumferential flange and the base plate and a radial dimension extending between an inner and an outer radius of the connecting ring; characterised in that the radial dimension in at least one area is smaller than the axial dimension. This provides for a simple construction whereby the connecting ring can be provided.

Due to the relative axial and radial dimensions of the connecting ring, circumferential flange may move radially relative to the base plate. Naturally, it will be appreciated that the more flexible the connecting ring is, the more free is the flange and the base plate to move relative to each other.

It should be understood that the connecting ring does not necessarily extend orthogonal between the base plate and the circumferential flange, but may form an angle. Such an angle may for example be provided due to production tolerances or for example if the connecting ring is injection moulded together with either the circumferential flange or the base plate in order to easy remove the injection moulded product from the mould. Furthermore such an angle can provide some additionally support and/or allowing a smooth transition between the base plate and the circumferential flange reducing the risk of settlement of solid matter. Such an angle between the connecting ring and the base plate will typically be between 30 and 90 degrees, alternatively between 40 and 80 degrees and especially between 45 and 60 degrees.

In the use situation such relative movement decreases the risk of detachment of the base plate from the skin of the user. Accordingly, the risk of leaks of skin-harming solid matter is reduced and the patient may use the ostomy carrying device for a longer period of time.

By providing an connecting ring flush with an inner surface of which extends in the axial direction, it may be avoided that solid matter can be trapped in the ostomy carrying device. This is more hygienic than devices wherein the connecting ring extends in the radial direction (or more in the radial direction than in the axial direction) whereby solid matter may be trapped between the connecting ring and base plate and/or the circumferential flange.

In one embodiment there may be provided more than one connecting ring. As an example, there may further be provided an outer connecting ring co-extending the aforementioned connecting ring, which in the following is called the inner connecting ring. The outer connecting ring may be concentric with the inner connecting ring i.e. having the same centre. However in other embodiments the centre of the two rings do not coincide. In yet another embodiment there may be provided a plurality of concentric connection rings, such as three, four or five.

An advantage of providing a plurality of connecting rings is that the axial strength may be improved while maintaining the relative radial freedom of movement between the base plate and the circumferential flange.

Alternatively, the outer radially flexible connecting ring or one of them may be provided as a plurality of connecting elements e.g. cylindrical shaped, which interconnect the flange and the base plate e.g. in a ring shaped zone having an inner diameter being larger than the outer diameter of the inner radially flexible connecting ring.

At least one of the connecting ring(s) may comprise an elastomer. It will be appreciated that the more elastic the connecting rings, the higher is the relative radial freedom of movement between the base plate and the circumferential flange. In the context of the present invention the term elastomer defined as a material which at room temperature can be stretched repeatedly and upon immediate release of the stress, will return to its approximate original length and shape.

It has been found that elastomers such as polyolefin elastomers in the form of copolymers of ethylene and octene or a foamed thermoplastic elastomer material in the form of a moulded foam product or an integral foam may be used for this purpose. A preferred such elastomer is Engage® 8401 Polyolefin Elastomer from DuPont Dow. Such copolymers may be injection moulded and compression moulded, has a low density reducing the weight of the product and show a suitable stiffness for the purpose of the present invention.

In one embodiment the radial dimension at any area of at least one of the connecting rings is smaller than its axial dimension. The axial dimension of at least one of the connecting rings may coincide with a normal of the circumferential flange and/or the base plate. Accordingly, the axial dimension of the connecting ring is substantially orthogonal to a plane generally defined by the base plate and/or the circumferential flange.

In order to facilitate laser welding of the connecting ring(s) and the base plate and/or the circumferential flange their materials may be chosen such that an absorption coefficient of at least one of the connecting rings at a predetermined optical wavelength is higher than an absorption coefficient of the circumferential flange and/or the base plate at said wavelength. By choosing one material with a low coefficient of absorption it may be possible for a laser beam to pass through the material and reach the material with the high coefficient of absorption such that said materials may be welded together at a transition zone.

The ostomy carrier device may be made as an one-component device i.e. in one piece. Alternatively, the device may be made as a two-component device wherein the circumferential flange and the radially flexible connecting ring(s) are made in one piece and connected to the base plate or the base plate and the connecting ring(s) are made in one piece and connected to the base plate. In yet another embodiment, the base plate, the connecting ring(s) and the circumferential flange are made as separate elements and attached to each other e.g. by means of laser welding.

As an alternative or a supplement to the laser welding the elements may be attached to each other in the injection moulding process by means of the so called two-component or three component process.

The axial dimension of at least one of the connecting rings may be at least two times the radial dimension, such as at least three times, such as at least four times, such as at least five times, such as at least ten times. It will be appreciated that the longer the axial dimension is compared to the radial dimension the easier it may be for base plate and the circumferential flange to move relative to each other.

The radial dimension of at least one of the connecting rings may change in the axial dimension. Accordingly, a cross-section of at least one of the connecting rings may be triangular. The top of the triangle may be attached to the base plate and the bottom of the triangle may be attached to the circumferential flange, or vice versa. Alternatively, the connecting ring(s) may have a shape that corresponds to two triangles, wherein the base parts are attached to each other (such that the two triangles together define a diamond). Naturally, two connecting rings need not the have the same cross-sectional shape.

In yet another embodiment the at least one connecting ring is formed of a foam. The structure, such as the walls in the foam is typically resilient giving the foam its spring-like characteristics, allowing it to be compressed and subsequently expand. Preferably the foam is a low density foam, typically having a density between 25-100 kg/m³. The foam can for example be a cross linked polyolefin foam, which is a foam which can be easily welded to the flange and the base plate. Alternatively in another embodiment the foam can be a polyurethane foam.

Preferably the axial dimension of the foam is between 1-5 millimeters in order to obtain the desired flexibility while at the same time maintaining a discrete ostomy carrier device. However, other dimensions can be provided within the scope of the invention.

At least one of the connecting rings may have an elongation at break of at least 300%, such as 500%, such as 1000%, such as 1500%.

At least one of the connecting rings may have a tensile strength between 10 and 50 MPa, such as 20 MPa, such as 30 MPa, such as 40 MPa.

At least one of the connecting rings may have a shore-A-hardness between 45 and 100 such as 50, such as 60, such as 70, such as 80, such as 90

At least one of the connecting rings may have a modulus of elasticity between 1 and 10, such as 2 such as 4, such as 6 such as 8.

The circumferential flange may be flexible. As an example an outer portion of the circumferential flange may be more flexible than the inner portion of the circumferential flange. The inner and outer portion of the circumferential flange may be free to move relative to the base plate.

According to a SECOND aspect the present invention relates to a method of making an ostomy carrier device according to the first aspect of the present invention, the method comprising the step of: attaching at least one of the connecting rings to the base plate; and attaching said connecting ring(s) to the circumferential flange.

The step of attaching the connecting ring may comprise the steps of welding the connecting ring(s) to the base plate and/or the circumferential flange comprises the step of welding the connecting ring(s) by means of laser welding and/or ultrasonic welding and/or heat welding and/or pulse welding.

The step of attaching the connecting ring may comprise the steps of welding the connecting ring(s) to the base plate and/or the circumferential flange comprises the step of welding the connecting ring(s) by means of two component injection moulding.

The following description relate to the first and second aspect of the present invention.

In the context of the present invention the term “circumferential” implies only that a member provides a structure surrounding an opening; such a member may have circular inner and outer edges, however, it could have any combination of, for example, round, oval or angular shapes.

When it is stated that an outer portion of the flange is free to move with respect to the base plate, this means that only an inner edge or a part of the inner portion of the flange is attached to the base plate, while the remainder of the flange is free to move relative thereto.

When the carrier device defines a general plane of reference, it is clear to the skilled person that no strictly mathematical plane is defined but a plane represented by the carrier device itself, such a device having a generally planar configuration. When the body side member is attached to the user, the plane of reference will be substantially parallel with the user's skin.

When it is stated that the connecting ring is radially flexible it should be understood that a point on the ring can be displaced transverse to the axial direction, i.e. the direction of the axial dimension, of the connecting ring when a force is applied thereto and that the point will return to, or approximately to its point of origin when the said force is removed.

The present invention is based on the recognition that the different portions of the mounting flange serve different specialised functions. More specifically, it has been found by the present inventors that the innermost portion of the flange should be adapted to withstand movements as well as deformation of the flexible adhesive base plate in order to prevent pealing of the bond to start from the inner edge, whereas the outermost portion of the flange should be adapted to withstand the external forces applied to the bond due to, primarily, movements of the collection bag which arise during use thereof. Indeed, in combination the inner and outer portions of the flange should ensure against axially directed pulling forces applied on the bag.

A first choice to protect a flexible inner portion of a coupling flange against excessive deformation originating from the adhesive base plate would be to make the latter thicker and stiffer. Such a solution can be easily implemented and is found to work well, the relatively stiff base plate protecting the mounting flange from “curling”. However, the present inventors have found that this solution will only work for a relatively short period of time after which peeling will start from the inner edge of the bond. The reason for this has been found to be due to long-term (i.e. over days) plastic deformation, or “creeping”, of the adhesive base plate material normally used for this purpose.

When the outer portion of the flange is more flexible than the inner portion of the flange, the outer peripheral portion of the flange can absorb movements between the carrier device and the collecting bag thus protecting against peeling of the bond between the flange members as well as providing a cushioning effect against forces acting on the periphery of the flanges, whereas the inner less flexible portion is adapted to withstand deformations originating from the adhesive pad. Preferably the flange is a relatively thin, substantially planar sheet or foil-like structure.

Although it is considered that the expression “more flexible” is clear to the skilled person within this technical field, i.e. properties with regard to flexibility which meet the stated objects of the invention, it could be defined that the average flexibility of the outer portion is at least 10%, preferably at least 20% higher than the average flexibility of the inner portion. Such an “average flexibility” would be relevant as, for example, the outer portion may comprise segments, e.g. ring-formed, which would locally be less flexible than the outer portion in general. It may very well be that for a given carrier device a difference of only 10% will solve the stated problem, whereas for another carrier device a difference of more than 100% will be necessary. Indeed, for a flange made of a single material, the above considerations would apply to the thickness of the different portions of the flange, i.e. the average thickness of the outer portion is correspondingly at least 5%, preferably at least 10% less than the average thickness of the inner portion. According to a preferred embodiment of the invention, a hinge is formed between the outer and inner portions of the flange, such a hinge providing an “un-coupling” of the inner and outer portions allowing the two portions to serve their respective purposes even better. More specifically, a hinge will be able to distribute the forces acting on the inner portion of the flange, and thus the stoma, when the outer portion is subject to external forces during use, this providing increased carrying comfort. Further, the hinge will be able to reduce the amount of deformation which is transferred from the inner to the outer portion of the flange, this reducing the risk that peeling starting from the inner edge of the flange will spread to the outer portion and ultimately to the outer edge of the flange, which then would amount to a leak being formed. The hinge may be formed with a thickness, which is less than the thickness of the inner and outer portions of the flange to which it connects, or it may be formed by a material having a desired high flexibility.

According to a further preferred embodiment, the flange has a coupling surface facing away from the base plate and defining an attachment area, an outer circumferential portion of the flange corresponding to at least 50% of the area being free to move with respect to the other members of the carrier device. This construction allows an adhesive coupling member of a collecting bag to be fastened primarily or entirely on this free portion, which ensures adequate protection against peeling action as well as against forces applied to the collecting bag that are transmitted directly to the base plate.

The invention will now be described in further detail below with reference to the schematic drawing, in which

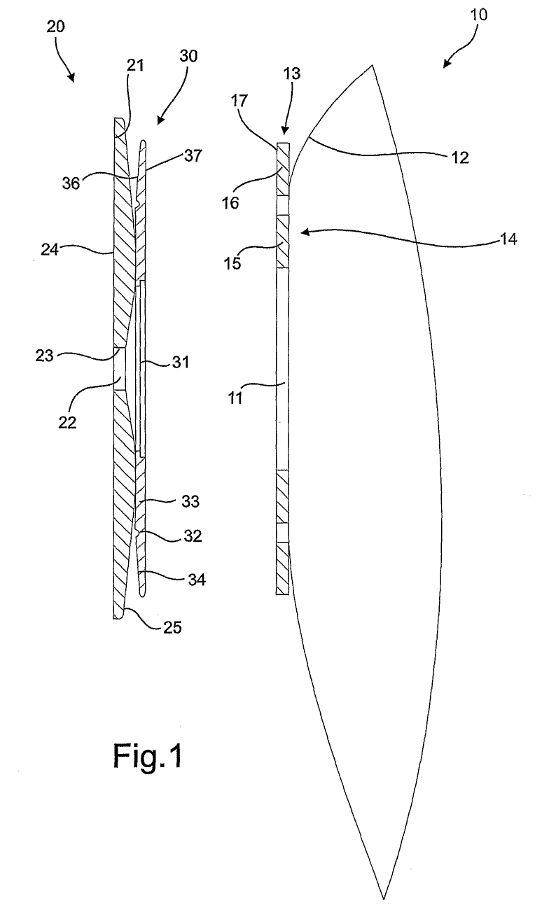

FIG. 1 shows an ostomy collecting system known in the art,

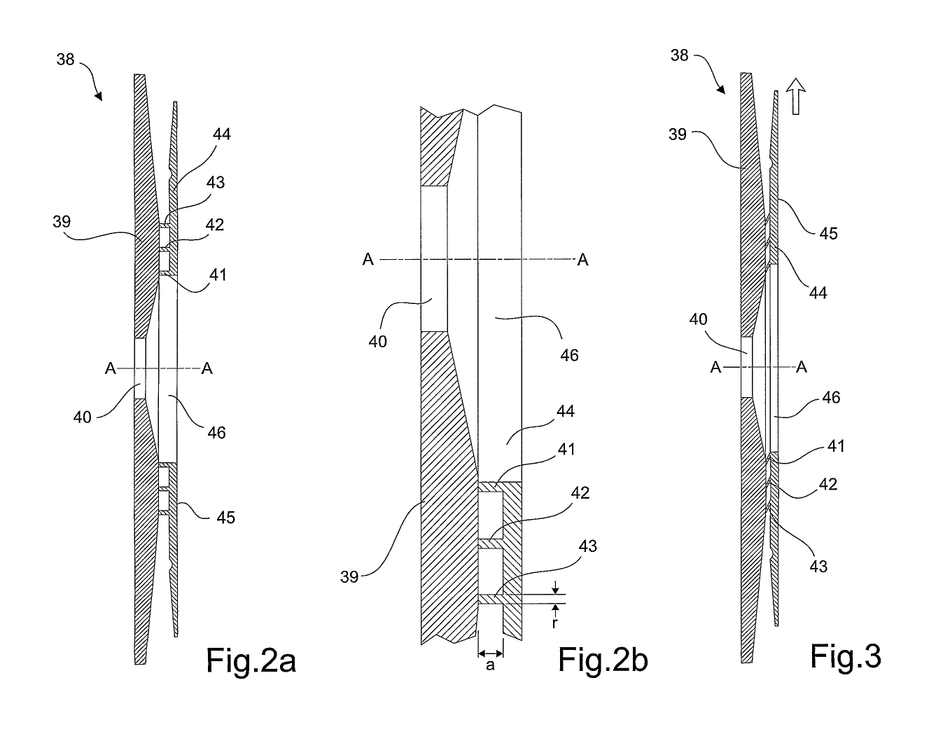

FIGS. 2a and 2b shows one embodiment of an ostomy carrier device according to the invention, and

FIG. 3 shows the embodiment of an ostomy carrier device as shown in FIGS. 2a and 2b in a loaded position.

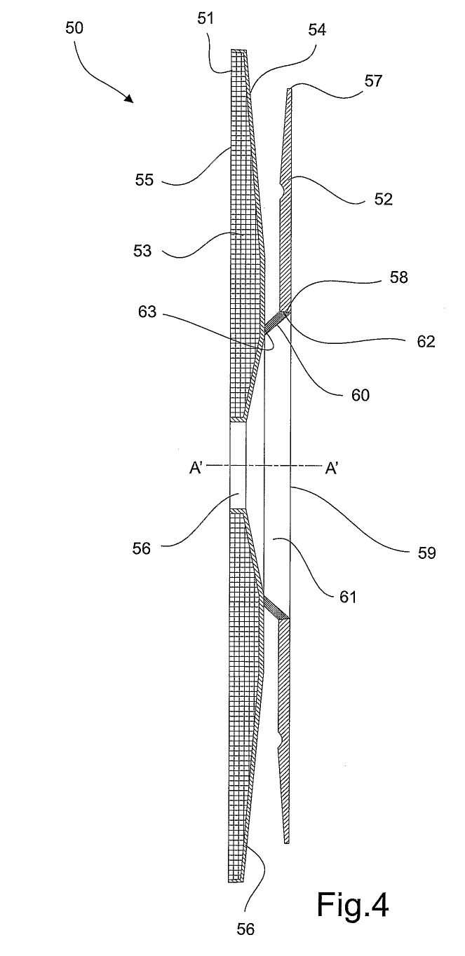

FIG. 4 shows another embodiment of an ostomy carrier device according to the invention seen in cross-section.

An ostomy collecting system as known from the art is shown in FIG. 1 and comprises a collecting bag 10 for collection of faeces and a carrier device 20 with a circumferential flange 30 for fastening the bag 10 around an intestinal orifice in the form of a so-called stoma in a user's abdominal wall (not shown), and for this purpose it has an inlet opening 11 in a bag wall 12. The bag 10 may either be closed at the bottom as shown or configured with a drainage device for discharging its contents. The inlet opening 11 is surrounded by connecting means for connecting the bag 10 with the flange 30 of the carrier device 20.

In the shown embodiment, the connecting means on the bag 10 is in the form of a second flange or bag flange 13 which is fastened to the bag wall 12 by means of a substantially circumferential attachment means defining an attachment zone 14 in such a manner that a rim portion of the bag flange 13 protrudes outwardly beyond the attachment zone. The bag flange 13 may be fastened to the bag wall 12 through any suitable means, for example directly by heat or UV welding or by means of an adhesive. In the shown embodiment the bag flange comprises two concentric rings 15, 16 connected to each other only at a number of narrow bridges (not to be seen in the figure). The two rings are adapted for mounting on an inner respectively an outer portion of the flange 30 and allow the bag flange to flex easily together with the hinge formed between the inner and outer portions of the flange 30. On the side facing away from the bag, the flange 13 is coated across substantially its entire surface with a thin layer 17 of adhesive, which may, for example, be a hydrogel adhesive, an acrylate adhesive or an adhesive of the hot-melt type. The layer 17 of adhesive is applied thinly, partly to keep down the thickness, partly to maintain the flexibility and resilience of the bag flange 13. This application may be effected, for example, by coating, spraying or application in a suitable pattern. In the delivery state of the bag, the layer 17 of adhesive is typically protected by a release liner (not shown) covering the layer.

The carrier device comprises a circumferential base plate 21 with an opening 22 and an inner free edge 23, and is designed to be adhered to the user's skin by means of a skin-friendly adhesive applied on the lower surface 24 of the base plate intended for attachment to the skin of the user. Preferably the peripheral portion of base plate is formed with a reduced thickness in order to better comply with movements and folding of the skin surface of the patient. The base plate 21 carries a flange or base plate flange 30 on its opposite upper surface 25, in which is formed an opening 31 providing communication between the opening 11 in the bag and the opening 22 in the base plate when the bag is mounted on the carrier device. The flange has a first surface 36 facing towards the base plate, a second opposite surface 37 facing away from the base plate, the second surface of the flange being adapted for removable and adhesive connection with the ostomy collecting bag. In the shown embodiment a hinge 32 is formed between an inner portion 33 and an outer portion 34 of the flange. In the shown embodiment, the different portions of the flange 30 is manufactured from the same material, and the hinge is formed with a thickness, which is less than the thickness of the inner and outer portions of the flange to which it connects. In order to provide a higher flexibility of the outer portion as compared with the inner portion, the outer portion is formed with a reduced thickness as compared with the inner portion. The flange is attached to the base plate corresponding to the reduced-thickness innermost edge portion of the flange, the reduced thickness providing improved heat transfer during the heat bonding process. Indeed, the flange may be attached by any suitable means just as the bond formed between the base plate and the flange may correspond to a smaller or larger area of the inner portion of the flange.

As it appears, the flange 30 is mounted at a distance from the opening 22, allowing the base plate portion surrounding the opening to be cut to provide an opening of a desired size in order to closely fit around the stoma of the user. In the delivery state of the carrier device, the adhesive lower surface 24 of the base plate is typically protected by a release liner (not shown) covering the layer. In the shown embodiment, the flange 30 is non-adhesive; however, the carrier device may also be provided with an adhesive base plate flange 30 adapted to match a non-adhesive bag flange 13, in which case a release liner would be provided on the base plate flange. Recently, adhesives have come on the market that are applied to both surfaces, but are before attachment non-sticking, thereby alleviating the necessity for a release liner.

FIG. 2a shows one embodiment of an ostomy carrier device 38 according to the invention.

The carrier device consists of an adhesive base plate 39. The adhesive base plate is adapted to be placed on the skin of a user around the stoma (not shown). A first opening 40 is arranged in the base plate for receiving the stoma. The adhesive base plate can for example be formed of a hydrocolloid.

A first connecting ring 41, a second connecting ring 42 and a third connecting ring 43 couples the adhesive base plate to a circumferential flange 44. The circumferential flange is formed with an adhesive surface 45 for connection with an ostomy bag (not shown). A second opening 46 is formed in the circumferential flange for receiving the stoma.

The connecting rings and the circumferential flange are moulded as one part during production. However, they could be formed as separate parts and then later attached to each other by for example welding or gluing. Alternatively the rings could be moulded together with the base plate.

FIG. 2b shows an enlarged section of the embodiment of the ostomy carrier device 38 illustrated in FIG. 2a. The section illustrates better the dimensions and proportions the connecting rings 41,42,43.

The third connecting ring has a radial dimension r, extending between the inner and outer radius of the connecting ring. Furthermore the third connecting ring has an axial dimension a, which extends in an axial direction along axis A-A between the circumferential flange 44 and the base plate 39.

As can be seen is the radial dimension r smaller than the axial dimension a. This relation is the same for the first and second connecting ring. However in alternative embodiments the specific dimensions may vary and differ from each connecting ring.

The mentioned relation between the radial dimension r and the axial dimension a provides that the connecting rings can be easily deformed, especially in a direction transverse to the axis A-A. This is illustrated in FIG. 3 where a force indicated by the open arrow influences the circumferential flange and the flange is consequently moved relative to the base plate 39 due to the deformation of the connecting rings.

As described earlier can the possible radial movement of the circumferential flange be increased further by forming the connecting rings of an elastic material. This allows for stretching of the connecting rings.

While in the foregoing embodiment of the invention has been disclosed in detail for purpose of illustration, it will be understood by those skilled in the art that many of these details may be varied without departing from the scope of the present invention as defined in the accompanying claims.

It can for example be provided any number of connecting rings, for example on or two, or any number above three. Furthermore can the relation of the radial dimension and the axial dimension vary from different connecting rings provide on the same ostomy carrier device and the dimensions may also vary on the individual connecting ring. Thus, it is within the scope of the invention that in areas of a connecting ring the radial dimension is larger than the axial direction, as long as the radial dimension in at least one are is smaller than the axial direction as described.

Furthermore can the circumferential flange be formed in a number of different ways and does not necessarily have to be adhesively attachable to the ostomy bag as illustrated. Such other circumferential flanges can for example be attachable to the ostomy bag via e.g. locking rings.

FIG. 4 shows another embodiment of an ostomy carrier device 50 according to the invention. The ostomy carrier device 50 is formed of an adhesive base plate 51 and a flange 52.

The adhesive base plate 51 is formed of a hydrocolloid adhesive 53 which is disposed on a backing layer 54 formed of a polyurethane film (not shown). Thus the adhesive 53 is exposed on the distal side 55 of the base plate 51 allowing the base plate to be adhered to the skin of an user (not shown).

The adhesive base plate has an annular periphery, e.g. circular or oval. However, the periphery may also form a square, triangle or any other geometrical shape.

A first through going hole 56 is provided in the base plate, having an axis A′-A′. The hole is adapted to receive a stoma when the base plate is attached to the skin of a user. Additionally the hole may be widened for example by cutting out some of adhesive plate surrounding the hole with a pair of scissors. Thus the size of the hole may be adapted to accommodate different sized and shaped stomas.

On the distal side 56 of the base plate the flange 52 is attached. The flange 52 is formed of a polymer and has a thickness, which in this embodiment makes it relative flexible, i.e. it may easily be flexed using hands. However, many other factors may also influence the flexibility of a material such as tension, humidity and temperature.

The flange has an outer annular periphery 57 and an inner annular periphery 58. The inner annular periphery defines a second through going hole 59. The flange is attached to the base plate via an elastic connecting ring 60. The connecting ring defines a third through going hole 61.

A distal end 62 of the connecting ring is welded together with the flange 52 along the inner annular periphery 58 and a proximal end 63 of the connecting ring is welded together with the backing layer of the base plate. When the connecting ring is welded together with the base plate and the flange to form the ostomy carrier device 50, the three part are arranged so that the first, second and third through going holes are coaxially aligned along the axis A′-A′.

It should be noted that the connecting ring and the backing layer is not necessarily welded together, in other embodiments it may be glued or otherwise joined together by any suitable method known in the art.

As can be seen in FIG. 4 the connecting ring 60 tapers from the distal end 62 towards the proximal end 63. In other words the radius of the distal end is larger than the radius of the proximal end. This provides a smooth surface, which reduces the risk of catching and colleting particles, keeping the ostomy carrier device clean and hygienic during use. Thus, for example when the user changes bags collected matter from stools will not end up contaminating the fresh bag or the users hands, or interfere with the coupling mechanism between the flange and the bag whether this coupling be adhesive coupling or mechanical coupling.

The connecting ring 60 is formed of a resilient and elastic material, having a low degree of visco-elasticity. Thus, when the connecting ring is deformed, for example the distal end is displaced radially relative to the proximal end it will very quickly return to its original position as soon as the stress deforming the connecting ring is removed. Materials suitable for forming such an elastic material being resilient and having a low visco-elasticity may be found within the group of elastomers. It has been found that in one embodiment especially thermoplastic elastomers (such as the ones described previously) bare well-suited and inhabits the above characteristics, in particular thermoplastic polyolefins. Such elastomers will be able to return from a displaced, deformed position to a neutral, original position in under a second when stress, such as a force applied to it, is removed.

1: An ostomy carrier device (38) comprising:
a base plate (39) for securing the carrier device (38) to a user's skin around a stoma, the base plate (39) having an opening (40) for receiving the stoma;
a circumferential flange (44) adapted for connection with and removal of an ostomy collecting bag, the circumferential flange (44) having an opening (46) for receiving a stoma;
at least one connecting ring (41,42,43) arranged between the circumferential flange (44) and the base plate (39),
at least one point on said connecting ring being radially displaceable from a neutral position, where no force is applied to the connecting ring, to a displaced position when force is applied to said at least one point, and that when said at least one point is in the displaced position said at least one point will move towards the neutral position when no force is applied to said connecting ring.

2: An ostomy carrier device according to claim 1, wherein the at least one connecting ring is formed of an elastic material, having a resilience allowing the at least one point to move from the displaced position towards the neutral position when no force is applied to the connecting ring.

3: An ostomy carrier device according to claim 1, wherein the at least one connecting ring is radially flexible.

4: An ostomy carrier device according to claim 1, wherein the at least one connecting ring (41,42,43) comprises an axial dimension (a) extending between the circumferential flange (44) and the base plate (39) and a radial dimension (r) extending between an inner and an outer radius of the at least one connecting ring (41,42,43); wherein in the radial dimension (r) in at least one area is smaller than the axial dimension (a).

5: An ostomy carrier device (38) according to claim 1, further comprising an outer connecting ring (42,43) co-extending an inner connecting ring (41,42).

6: An ostomy carrier (38) device according to claim 1, wherein the at least one connecting ring (41,42,43) comprises an elastomer.

7: An ostomy carrier device (38) according to claim 4, wherein the radial dimension (r) at any area of at least one of the connecting rings (41,42,43) is smaller than its axial dimension (a).

8: An ostomy carrier device (38) according to claim 1, wherein an absorption coefficient of at least one of the connecting rings (41,42,43) at a predetermined optical wavelength is higher than an absorption coefficient of the circumferential flange (44) and/or the base plate (39) at said wavelength.

9: An ostomy carrier device (38) according to claim 4, wherein the axial dimension (a) of at least one of the connecting rings (41,42,43) is at least two times the radial dimension (r).

10: An ostomy carrier device (38) according to claim 4, wherein the radial dimension (r) of at least one of the connecting rings (41,42,43) changes in the axial dimension (a).

11: An ostomy carrier device according to claim 1, wherein the at least one radially flexible connecting ring is formed of a foam.

12: An ostomy carrier device according to claim 11, wherein the foam has a density between 25-100 kg/m³.

13: A method of making an ostomy carrier device (38) according to claim 1, the method comprising the step of:
welding the at least one connecting ring(s) (41,42,43) to the base plate (39); and
welding said connecting ring(s) (41,42,43) to the circumferential flange (44).

14: A method according to claim 13, wherein the steps of welding the connecting ring(s) (41,42,43) to the base plate (39) and/or the circumferential flange (44) comprises the step of welding the connecting ring(s) (41,42,43) by means of laser welding.
